(19) United States
(12) Patent Application Publication
(10) Pub. No.: US 2019/0053527 A1
(43) Pub. Date: Feb. 21, 2019

(54) METHOD FOR PREPARING A PROBIOTIC POWDER USING A TWO-IN-ONE WHEY-CONTAINING NUTRIENT MEDIUM

(71) Applicants: INSTITUT NATIONAL DE LA RECHERCHE, AGRONOMIQUE (INRA), Paris (FR); INSTITUT SUPERIEUR DES SCIENCES AGRONOMIQUES, ARGOALIMENTAIRES, HORTICOLES ET, Rennes (FR)

(72) Inventors: Romain JEANTET, Rennes (FR); Song HUANG, Suzhou (CN); Gwénaël JAN, Rennes (FR); Pierre SCHUCK, Chartres De Bretagne (FR); Yves LE LOIR, Le Rheu (FR); Xiao-Dong CHEN, Suzhou (CN)

(21) Appl. No.: 15/761,519

(22) PCT Filed: Sep. 20, 2016
(86) PCT No.: PCT/EP2016/072324

§ 371 Date: Mar. 20, 2018

(30) Foreign Application Priority Data
Sep. 21, 2015 (EP) 15306465.4

(51) Int. Cl. A23L 33/19; A23L 33/195; A23P 10/40; A23L 29/00; A23L 33/135; A61K 35/745; C12N 1/04

(52) U.S. Cl. CPC A23L 33/19; A23L 33/195; A23P 10/40; C12N 1/04; A23L 33/135; A61K 35/745; A23L 29/065

(57) ABSTRACT

The present invention relates to a method for preparing a probiotic powder comprising at least one probiotic bacterium, said method comprising: a) providing a probiotic biomass composition comprising at least one probiotic bacterium resulting from the culture of said probiotic bacterium in a whey-containing nutrient medium having a total solid content ranging from above 25% by weight to up to 35% by weight, based on the total weight of the said whey-containing nutrient medium; b) spray drying or freeze drying the said probiotic biomass provided at step a) so as to obtain the said probiotic powder.

### FIELD OF THE INVENTION

The present invention relates to the field of manufacturing probiotic-containing compositions.

### BACKGROUND OF THE INVENTION

According to the definition of FAO/WHO, probiotics consist in viable microorganisms that confer a health benefit to the host when administered in adequate amounts (FAO/WHO, 2001). Nowadays, food products that incorporate probiotics are usually categorized as functional foods due to the increasing evidences of the health benefits reported in both in vitro and in vivo experiments (Kamada et al., 2013; Pessione, 2014; Rodgers, 2008). For instance, Propionibacterium freudenreichii probiotic potential includes immunomodulation by P. freudenreichii strain ITG 20 via key surface proteins (Cousin et al., 2012; Cousin et al., 2010; Foligné et al., 2010; Foligné, et al., 2013). Similarly, Lactobacillus casei BL23 is an anti-inflammatory strain, also known to attenuate colitis.

As a consequence of the increasing demand of these probiotic foods in the recent decades, the current scientific and technical bottleneck addresses the preservation of the probiotic viability during production process, long-term storage and digestion.

Drying of bacteria has been recognized as an efficient approach to stabilize the bacterial cells and prolong the shelf life of biological products. Besides, the drying matrix may encapsulate the probiotic cells after drying, which has been proven to exert a protective effect against the adverse conditions during storage and delivery in the digestive tracts (Cook et al., 2012; Paéz et al., 2012; Semyonov et al., 2010; Yonekura et al., 2014).

Up to date, freeze drying is still the most frequently used technique for drying of bacteria. Although the conditions (cold temperature, sublimation of water) used in this process are generally beneficial for maintaining the bacteria viability, its relatively high cost and low energy efficiency have been considered as a stumbling block when facing the increasing commercial demand of probiotic products (Holzapfel, 2014; Lievense & van′t Riet, 1993; Peighambardoust et al., 2011).

Alternatively to freeze drying, spray drying has been recognized as an efficient technique to produce large amount of dried probiotics. The advantages of spray drying mainly consist in the relatively low production cost, approximately 10 times cheaper than freeze drying, the ability in large-scale production and its mature application in dairy and pharmaceutical industries (Santivarangkna et al., 2007; Schuck et al., 2013). However, the challenge of utilizing spray drying to produce probiotic powders is primarily related to the existence of high temperature during process, which may cause irreversible damages to bacterial cells and may subsequently negatively impact their viability (Ananta et al., 2005; Fu & Etzel, 1995; Lievense & van′t Riet, 1994).

Extensive investigations have been carried out in order to improve the remaining viability of probiotic bacteria after spray drying and during following storage. The strategies include process optimization, application of protectants and enhancement of cellular resistance (Desmond et al., 2001; Fu & Chen, 2011; Liu et al., 2015; Peighambardoust et al., 2011). However, these strategies usually and merely focus on the drying step, rarely considering the overall process from growth to drying of bacteria. Therefore, standard laboratory media were used to grow bacteria in most studies (Barbosa et al., 2015; Golowczyc et al., 2010; Lavari et al., 2014; Lian et al., 2002; Perdana et al., 2013; Sunny-Roberts & Knorr, 2009).

For example, it was shown that MRS (de Man et al., 1960) and Yeast Extract Sodium Lactate (YEL) (Malik et al., 1968) broths represent generally standard growth medium of Lactobacillus sp. and Propionibacterium sp. respectively, corresponding to a total solid content (TS) of about 5 wt %. However, using standard growth conditions has several adverse consequences when using these media as raw materials in industrial spray drying: in particular, low powder flow rate, high evaporation rate and drying temperature needed, and occurrence of undesired caking or sticking in the relatively fine powders (Stoklosa et al., 2012; Wu et al., 2014). Conversely, an inhibition of the bacterial growth is expected when increasing the medium total solid (TS) content, because of the higher osmotic stress (Chirife et al., 1983; Vasseur et al., 1999; Walter et al., 1987).

When the probiotics cells have been grown in these standard nutrient media, the subsequent operations required before the accessing to the drying unit include rinsing, harvesting and re-suspending of bacteria. There are several disadvantages in this conventional process of spray drying of bacteria. From the practical point of view, the standard laboratory media used for growth of bacteria, e.g. the most frequently MRS, are normally expensive and non-food grade (De Man et al., 1960). In addition to the waste of material, the removal of these media may lead to the loss of bacteria viability and increase the risk of contamination during the intermediate operations such as rinsing, centrifugation and re-suspension. The possible residual components on the bacteria pellets from the media may also cause interference to the subsequent operation and further application.

Furthermore, documents such as FR 2 802 212, EP 0 818 529 and WO 01/36590 have disclosed the benefits of co-pulverization of probiotics obtained from standard growth together with protective agents, such as, sweet whey concentrate, concentrated milk, lactose, saccharose, trehalose, galactose, starch, sorbitol, casein, beta-lactoglobulin, alpha-lactalbumin, soy, serum albumin, glutenin, prolamin, lysine, cysteine, glycine, vitamins, in order to improve their survival after spray drying.

In the same line, Jantzen et al. (2013), discloses a spray drying method of L. reuteri comprised in a watery 20% whey solution complemented with 0.5% (w/v) of yeast extract.

Finally, R U 2010 140092 and CS 9 100 190 disclose a spray drying method of probiotics in the presence of 20-25% (w/v) whey.

Therefore, there is a need to provide new means to improve bacterial survival after a spray drying process.

There is also a need for simplifying the spray drying process.

### SUMMARY OF THE INVENTION

In one aspect, the invention relates to a method for preparing a probiotic powder comprising at least one probiotic bacterium, said method comprising:

a) providing a probiotic biomass composition comprising at least one probiotic bacterium resulting from the culture of said probiotic bacterium in a whey-containing nutrient medium having a total solid content ranging from above 25% by weight to up to 35% by weight, based on the total weight of the said whey-containing nutrient medium;

b) spray drying or freeze drying the said probiotic biomass provided at step a) so as to obtain the said probiotic powder.

In another aspect, the invention also relates to a probiotic powder comprising at least one probiotic bacterium obtained by a method according to the invention.

In another aspect, the invention also relates to the probiotic powder according to the invention for use for improving health of a human or an animal body.

In a still another aspect, the invention relates to the use of a whey-containing nutrient medium having a total solid content ranging from 15% by weight to 35% by weight, based on the total weight of the said whey-containing nutrient medium, for culturing at least one probiotic bacterium, for the preparation of a probiotic powder comprising the said at least one probiotic bacterium.

Another aspect of the invention relates to the use of a whey-containing nutrient medium having a total solid content ranging from 15% by weight to 35% by weight, based on the total weight of the said whey-containing nutrient medium, said whey from the whey-containing nutrient medium being the exclusive nitrogen source, for culturing at least one probiotic bacterium, for the preparation of a probiotic powder comprising the said at least one probiotic bacterium.

In another aspect, the invention relates to the use of a whey-containing nutrient medium having a total solid content ranging from above 25% by weight to up to 35% by weight, based on the total weight of the said whey-containing nutrient medium, for culturing at least one probiotic bacterium, for the preparation of a probiotic powder comprising the said at least one probiotic bacterium.

### LEGENDS OF THE FIGURES

FIG. 1. Plots illustrating the final bacteria populations (left Y axis) and the dependency (right Y axis; diamonds) of bacteria growth on casein peptone of (A) L. casei and (B) P. freudenreichii in the sweet whey nutrient media with different total solid content (TS) and with (dark gray bars) or without (light gray bars) supplement of casein peptone (in comparison to the standard media, i.e., MRS broth for L. casei and YEL broth for P. freudenreichii). Mean±SEM, n=6. Different letters: significantly different between the bacteria population (p<0.05); Different number of *: significantly different between the dependency (p<0.05).

FIG. 2. Plots illustrating the remaining bacteria populations (left Y axis; gray bars) and survival (right Y axis; diamonds) of (A) L. casei and (B) P. freudenreichii in the sweet whey media with different TS after spray drying; (C) Plot illustrating the survival of bacteria growing and spray drying in the 5% media (1), growing in 5% medium but being added to 30% before drying (2), growing and spray drying in 30% media (3). Mean±SEM, n=3. Different letters: significantly different between the bacteria population (p<0.05); Different number of *: significantly different between the survival (p<0.05).

FIG. 3. Plots illustrating the volume of particle size of the spray dried powders of (A) L. casei and (B) P. freudenreichii from the sweet whey media with different TS, 5% (curve 1), 10% (curve 2), 20% (curve 3), 30% (curve 4) and 40% (curve 5). The curves were obtained from the average value of duplicate experiments. The size range was measured from 0.1 to 2000 μm; the values of volume at the ranges from 0.1 to 1 μm and from 200 to 2000 μm were all 0.

FIG. 4. Plots illustrating the change of bacteria populations in the spray dried powders of (A) L. casei and (B) P. freudenreichii from the sweet whey media with different TS, 5% (diamonds), 10% (squares), 20% (triangles), 30% (large circles) and 40% (small circles), during storage at 4° C. for 120 days. Mean±SEM, n=3. (C) Plots illustrating the final log reduction of two probiotics strains L. casei (circles) and P. freudenreichii (squares) when storage for 120 days. Different number of *: significantly different between the survival (p<0.05).

### DETAILED DESCRIPTION OF THE INVENTION

The inventors have surprisingly found that it is possible to use a concentrated whey-containing nutrient medium to grow probiotic bacteria in order to achieve a two-step spray drying process comprising (i) growing the probiotics and (ii) drying said probiotics culture. The inventors have overcome the prejudices in the art acknowledging (1) the putative inhibition of probiotics growth when using a nutrient medium with high total solid content and (2) the need for artificially increasing the total solid content of the culture obtained with the use of “standard” growth conditions, just prior spray drying or freeze drying.

Furthermore, the inventors show for the first time that probiotic bacteria may be cultured in a whey-containing nutrient medium having a total solid content of whey of above 25 wt % and up to 35 wt %. Indeed, the skilled person in the art had the prejudice that a total solid content of whey up to 25 wt % could not be used to grow probiotics.

In addition, the inventors found that the whey may constitute the exclusive nitrogen source in the whey-containing nutrient medium, as to provide for the growth of the probiotic bacteria.

Method

In one aspect, the invention relates to a method for preparing a probiotic powder comprising at least one probiotic bacterium, said method comprising:

a) providing a probiotic biomass composition comprising at least one probiotic bacterium resulting from the culture of said probiotic bacterium in a whey-containing nutrient medium having a total solid content ranging from 15% by weight to 35% by weight, based on the total weight of the said whey-containing nutrient medium;

b) spray drying or freeze drying the said probiotic biomass provided at step a) so as to obtain the said probiotic powder.

Within the scope of the invention, a “probiotic powder” is intended to refer to a particulate product comprising live microorganisms that are intended to impart health benefits when consumed by a living animal or human body in adequate amounts.

In particular, health benefits may encompass the prevention or the treatment of e.g. digestive disorders, such as diarrhea, lactose intolerance, colitis, liver disease, irritable bowel syndrome and inflammatory bowel disease; allergic disorders, such as atopic dermatitis and allergic rhinitis; oral health problems. Some other benefits of probiotics also include immune stimulation, enhancement of bowel mobility, reduction of inflammatory reactions and even cancer apparition.

Within the scope of the invention, a “biomass composition” is intended to mean living probiotic microorganisms and/or any biological material derived from said living, or recently living probiotic microorganisms. A biomass composition according to the invention includes living probiotics together with unconsumed compounds from the nutrient medium and by-products of the probiotics metabolism.

Within the scope of the invention, the terms “culture” and “growth” are intended to be equivalent and may be therefore substituted to one another, and further refer to the step of multiplying viable probiotics to achieve a desired biomass composition.

Within the scope of the invention, a “nutrient medium” is meaning a nutrient composition comprising at least (i) one or more pH buffering system(s); (ii) inorganic salts; (iii) trace elements; (iv) free amino acids; (v) vitamins; (vi) one or more carbon/energy source(s) and suitable for cultivating at least one probiotic bacterium under appropriate growth conditions.

Within the scope of the invention, a “solid content” of any given compound in a mixture refers to the solid weight of said component as compared to the total solid (TS) weight of said mixture. In practice, the solid content of any given compound is expression in percent (%) by weight, as compared to the total weight of the said composition.

Within the scope of the invention, the expressions “a nutrient medium having a total solid content of ‘X’ wt %”, “a nutrient medium having a total solid content of ‘X’ % by weight” and “a nutrient medium having a total solid content of ‘X’ wt %, as compared to (or based on) the total weight of the said nutrient medium” are intended to be equivalent and may be substituted to one another.

In practice, the total solid (TS) content of an aqueous liquid substance may be measured by evaporating the liquid in an oven at a temperature of at least 100° C. for a sufficient time to collect a dry powder, which is subsequently weighted. The determined dried weight is then compared to the total weight of the liquid substance before evaporation.

Within the scope of the invention, “whey” is intended to mean, according to its conventional meaning in the art, the by-product substance originated from cheese production.

Probiotic Bacteria

Within the scope of the invention, the terms and expressions “probiotic”, “probiotic bacterium” and “probiotic microorganism” are intended to be equivalent and may be therefore substituted to one another.

In some embodiments, said probiotic bacterium is selected in a group comprising Bifidobacterium sp., Lactobacillus sp., Lactococcus sp., Propionibacterium sp., Streptococcus sp. and a mixture thereof.

In some embodiments, said probiotic bacterium is selected in a group comprising Bifidobacterium adolescentis, B. animalis, B. bifidum, B. breve, B. infantis, B. lactis, B. longum; Lactobacillus acidophilus, L. casei, L. delbrueckii, L. gasseri, L. paracasei, L. plantarum, L. reuteri, L. salivarius, L. rhamnosus, Lactococcus lactis, Propionibacterium freudenreichii, Streptococcus thermophilus and a mixture thereof.

In some embodiments, said probiotic bacterium is selected in a group comprising Lactobacillus acidophilus, L. amylovorus, L. brevis, L. casei, L. rhamnosus, L. crispatus, L. delbrueckii subsp. bulgaricus, L. fermentum, L. gasseri, L. helveticus, L. johnsonii, L. lactis, L. paracasei, L. plantarum, L. reuteri, L. salivarius, L. gallinarum and a mixture thereof.

In some embodiments, said probiotic bacterium is selected in a group comprising Bifidobacterium adolescentis, B. animalis, B. breve, B. bifidum, B. infantis, B. lactis, B. longum and a mixture thereof.

In some embodiments, said probiotic bacterium is selected in a group comprising Bacillus cereus, Clostridium botyricum, Enterococcus faecalis, Enterococcus faeciuma, Escherichia coli, Lactococcus lactis subsp. cremoris, Lactococcus lactis subsp. lactis, Leuconostoc mesenteroides subsp. dextranicum, Pediococcus acidilactici, Propionibacterium freudenreichii, Saccharomyces boulardii, Streptococcus salivarius subsp. thermophiles, Sporolactobacillus inulinus and a mixture thereof.

In some preferred embodiments, said probiotic bacterium is selected in a group comprising L. casei and P. freudenreichii.

In some embodiments, the said probiotic bacterium is a thermoresistant probiotic bacterium.

Within the scope of the invention, a thermoresistant probiotic bacterium is intended to refer to a probiotic bacterium that can sustain temperatures up to 60° C., preferably up to 55° C., without showing impaired survival.

In practice, a thermoresistant probiotic bacterium suitable for implementing the invention may be used in method for preparing cheese, in particular method comprising a step in which the thermoresistant probiotic bacterium is brought to temperature comprised from 45° C. to 60° C., preferably from 50° C. to 55° C.

In some embodiments, a thermoresistant probiotic bacterium suitable for implementing the invention may be used in method for yogurt, in particular method comprising a step in which the thermoresistant probiotic bacterium is brought to temperature comprised from 38° C. to 47° C., preferably from 40° C. to 45° C.

Growth Conditions

The inventors have shown that spray drying of a biomass comprising probiotics may be performed without the need of concentrating said biomass prior to achieving spray drying, as disclosed abundantly in the literature.

Therefore, the inventors disclose hereby a simple process to grow and dry the probiotics in sweet whey, which was chosen a two-in-one nutrient medium, rendering the steps such as rinsing, centrifugation and re-suspension obsolete.

Within the scope of the invention, a “two-in-one nutrient medium” is intended to mean a nutrient medium that is suitable to perform the step of growing the probiotic bacteria to obtain a probiotic biomass and further adapted to perform spray drying of said biomass, without the need of removing said nutrient medium from said probiotic bacteria after growth.

Within the scope of the invention, a total solid content ranging from 15% to 35% encompasses a total solid content of 16%, 17%, 18%, 19%, 20%, 21%, 22%, 23%, 24%, 25%, 26%, 27%, 28%, 29%, 30%, 31%, 32%, 33% and 34%.

In some embodiments, the whey-containing nutrient medium has a total solid content ranging from 20% by weight to 30% by weight, based on the total weight of the said nutrient medium.

In some other embodiments, the whey-containing nutrient medium has a total solid content ranging from above 25% by weight to up to 35% by weight, based on the total weight of the said nutrient medium.

Within the scope of the invention, a total solid content ranging from above 25% to up to 35% encompasses a total solid content of 25.5%, 26.0%, 26.5%, 27.0%, 27.5%, 28.0%, 28.5%, 29.0%, 29.5%, 30.0%, 30.5%, 31.0%, 31.5%, 32.0%, 32.5%, 33.0%, 33.5%, 34.0%, 34.5% and 35.0%. As shown in the example section here below, a biomass composition, resulting from a culture of said probiotic bacterium in a whey-containing nutrient medium having a total solid content ranging from 15 wt % to 35 wt %, preferably from above 25% to up to 35 wt %, presents a higher viability (as expressed in CFU·mL⁻¹) and a higher survival after spray drying, as compared to a biomass composition resulting from a culture of said probiotic bacterium in a whey-containing nutrient medium having a total solid content below 15 wt %, preferably strictly below 26%, or above 35 wt %.

As also shown in the example section here below, a biomass composition, resulting from a culture of said probiotic bacterium in a whey-containing nutrient medium having a total solid content ranging from 15 wt % to 35 wt %, presents a higher survival after spray drying, as compared to a biomass composition resulting from a culture of said probiotic bacterium in a whey-containing nutrient medium having a total solid content below 10 wt %, but which total solid content was adjusted to 15 wt % to 35 wt %, just prior to spray drying.

Without wishing to be bound by any particular theory, the inventors believe that the probiotic biomass obtained in whey-containing nutrient media with high total solid content is tolerant due to the cellular response, e.g., accumulation of intracellular compatible solutes triggered by the high osmolality during growth.

In some embodiments, the whey is sweet whey, which is a by-product of rennet-coagulated cheese. Sweet whey generally has a pH strictly above 5.1.

In some other embodiments, the whey is acid whey, which is a by-product obtained during the making of acid types of dairy products, such as cottage cheese or strained yogurts. Acid whey generally has a pH equal or inferior to 5.1.

In practice, sweet whey may originate from commercially available powder, such as e.g. Lactalis ingredients (France).

In some embodiments, whey-containing nutrient media may be prepared by pouring whey powders into any suitable liquid, such as deionized water, PBS buffer, or standard nutrient medium, such as e.g. MRS (Man Rogosa Sharpe) broth, M17 broth or YEL broth.

In certain embodiments, a whey-containing nutrient medium according to the invention may be prepared by pouring a whey powder into deionized water.

M17 broth is preferably used for the growth of Lactococcus sp. MRS broth or vegetable MRS broth is preferably used for the growth of Lactobacillus sp. YEL broth is preferably used for the growth of Propionibacterium sp.

Indeed, any culture medium known from the one skilled in the art may be used for growing the bacteria of interest, which include culture media based on milk, culture media based on milk permeate, culture media based on molasses or also culture media corn steep liquor.

In some embodiments, the whey-containing nutrient medium may further comprise one or more protective agent(s), known to have a beneficial effect on the survival of the probiotics after spray drying or freeze drying.

In some embodiments, said protective agent(s) may be chosen in a group comprising trehalose, saccharose, galactose, starch, sucrose, maltose, lactose, glucose, fructose, sorbitol, dextran, maltodextrin, skim milk (optionally under the form of a skim milk powder), betaine, galactomannane, carraghenane, pectin, casein, beta-lactoglobulin, alpha-lactalbumin, serum albumin, albumin, globulin, glutelin, prolamin, protamine, lysine, cysteine, glycine, glycerol, acacia gum (also termed “Arabic gum”) and a mixture thereof.

In some embodiments, casein may be provided in the form of casein peptone as a preferred nitrogen source.

In some certain embodiments, said nutrient medium may further comprise casein peptone.

In some embodiments, the casein peptone may represent from 0.1% to 1% (w/w) of the weight of the nutrient medium.

Surprisingly, the inventors have noticed that the whey comprised in the whey-containing nutrient medium may be the exclusive nitrogen source. As shown in the example section below, above a total solid content of whey of the whey containing medium of 20 wt %, as the total solid content of whey of the whey containing medium increases, the dependency of growth towards external additional nitrogen source decreases.

In other words, the whey-containing nutrient medium may be free or substantially free of an external additional nitrogen source.

Within the scope of the invention, the expression “substantially free of an additional nitrogen source” is intended to mean a whey-containing nutrient medium comprising strictly less than 2.5 wt % of an additional nitrogen source, preferably less than 1 wt %, more preferably less than 0.5 wt %, based on the total weight of the said nutrient medium.

In practice, an external additional nitrogen source may be selected in the group comprising yeast extract, beef extract, peptone, casein peptone and the like.

In some embodiments, the whey-containing nutrient medium is free or substantially free of yeast extract, beef extract, peptone, casein peptone and the like.

One aspect of the invention relates to a method for preparing a probiotic powder comprising at least one probiotic bacterium, said method comprising:
a) providing a probiotic biomass composition comprising at least one probiotic bacterium resulting from the culture of said probiotic bacterium in a whey-containing nutrient medium having a total solid content ranging from 15% by weight to 35% by weight, based on the total weight of the said whey-containing nutrient medium, said whey from the whey-containing nutrient medium being the exclusive nitrogen source;
b) spray drying or freeze drying the said probiotic biomass provided at step a) so as to obtain the said probiotic powder.

Another aspect of the invention relates to a method for preparing a probiotic powder comprising at least one probiotic bacterium, said method comprising:
a) providing a probiotic biomass composition comprising at least one probiotic bacterium resulting from the culture of said probiotic bacterium in a whey-containing nutrient medium having a total solid content ranging from above 25% by weight to up to 35% by weight, based on the total weight of the said whey-containing nutrient medium;
b) spray drying or freeze drying the said probiotic biomass provided at step a) so as to obtain the said probiotic powder.

In some embodiments, the whey-containing nutrient medium has a total solid content ranging from 26% by weight to 33% by weight, based on the total weight of the said whey-containing nutrient medium.

Another aspect of the invention relates to a method for preparing a probiotic powder comprising at least one probiotic bacterium, said method comprising:
a) providing a probiotic biomass composition comprising at least one probiotic bacterium resulting from the culture of said probiotic bacterium in a whey-containing nutrient medium having a total solid content ranging from above 25% by weight to up to 35% by weight, based on the total weight of the said whey-containing nutrient medium, said whey from the whey-containing nutrient medium being the exclusive nitrogen source;
b) spray drying or freeze drying the said probiotic biomass provided at step a) so as to obtain the said probiotic powder.

In practice, a pre-culture of at least one probiotic bacterium may be performed in an adequate nutrient medium, with or without whey, before inoculation in the final culture, intended to be spray dried. Such nutrient medium may be as already described above, e.g. M17 broth, MRS broth or YEL broth.

In some embodiments, the nutrient medium is free of yeast extract and/or yeast biomass.

Culture conditions with respect to the temperature, time and agitation may be conform to the culture conditions from the state of the art, depending of the type of probiotic.

Is some embodiments, the temperature of the probiotic culture is ranging from about 20° C. to about 40° C., preferably ranging from about 30° C. to about 37° C.

Is some embodiments, a temperature of the probiotic culture ranging from about 20° C. to about 40° C. includes a temperature of about 21° C., 22° C., 23° C., 24° C., 25° C., 26° C., 27° C., 28° C., 29° C., 30° C., 31° C., 32° C., 33° C., 34° C., 35° C., 36° C., 37° C., 38° C. and 39° C.

In some embodiments, the time length of the probiotic culture is ranging from about 24 h to about 150 h, preferably from about 48 h to about 120 h.

In some embodiments, a time length of the probiotic culture ranging from about 24 h to about 150 h includes a time length of about 24 h, 30 h, 36 h, 42 h, 48 h, 54 h, 60 h, 66 h, 72 h, 78 h, 84 h, 90 h, 96 h, 102 h, 108 h, 114 h, 120 h, 126 h, 132 h, 138 h, 144 h, 150 h.

Biomass Composition

In contrast to previously known methods for preparing probotic-containing powders wherein the biomass composition comprising probiotics that are undergoing spray drying, the method described herein does not comprise a step wherein the final total solid (TS) content is adjusted just prior to spray drying.

According to the method described herein, the final total solid (TS) content may be adjusted during the preparation of the whey-containing nutrient medium, i.e. prior to starting the culture of the probiotic bacterium itself.

As mentioned above, the probiotic biomass composition to be spray dried according to the invention does not require any concentration step in order to artificially control the concentration of probiotic cells and/or the total solid content. In particular, the biomass composition to be spray dried according to the invention does not require any sedimentation, or centrifugation step.

In some embodiments, a biomass composition to be spray dried according to the invention may comprise at least 5×10⁷ CFU·mL⁻¹ of said at least one probiotic bacterium.

In some embodiments, a biomass composition comprising at least 5×10⁷ CFU·mL⁻¹ of said at least one probiotic bacterium includes 6×10⁷ CFU·mL⁻¹, 7×10⁷ CFU·mL⁻¹, 8×10⁷ CFU·mL⁻¹, 9×10⁷ CFU·mL⁻¹, 1.0×10⁸ CFU·mL⁻¹, 1.2×10⁸ CFU·mL⁻¹, 1.4×10⁸ CFU·mL⁻¹, 1.6×10⁸ CFU·mL⁻¹, 1.8×10⁸ CFU·mL⁻¹, 2.0×10⁸ CFU·mL⁻¹, 2.2×10⁸ CFU·mL⁻¹, 2.4×10⁸ CFU·mL⁻¹, 2.6×10⁸ CFU·mL⁻¹, 2.8×10⁸ CFU·mL⁻¹, 3.0×10⁸ CFU·mL⁻¹, 3.5×10⁸ CFU·mL⁻¹, 4.0×10⁸ CFU·mL⁻¹, 4.5×10⁸ CFU·mL⁻¹, 5.0×10⁸ CFU·mL⁻¹, 5.5×10⁸ CFU·mL⁻¹, 6.0×10⁸ CFU·mL⁻¹, 6.5×10⁸ CFU·mL⁻¹, 7.0×10⁸ CFU·mL⁻¹, 7.5×10⁸ CFU·mL⁻¹, 8.0×10⁸ CFU·mL⁻¹, 8.5×10⁸ CFU·mL⁻¹, 9.0×10⁸ CFU·mL⁻¹, 9.5×10⁸ CFU·mL⁻¹, 10⁹ CFU·mL⁻¹, 2×10⁹ CFU·mL⁻¹, 3×10⁹ CFU·mL⁻¹, 4×10⁹ CFU·mL⁻¹, 5×10⁹ CFU·mL⁻¹, 6×10⁹ CFU·mL⁻¹, 7×10⁹ CFU·mL⁻¹, 8×10⁹ CFU·mL⁻¹, 10¹⁰ CFU·mL⁻¹, 2×10¹⁰ CFU·mL⁻¹, 3.10¹⁰ CFU·mL⁻¹, 4×10¹⁰ CFU·mL⁻¹, 5×10¹⁰ CFU·mL⁻¹, 6×10¹⁰ CFU·mL⁻¹, 7×10¹⁰ CFU·mL⁻¹, 8×10¹⁰ CFU·mL⁻¹, 9×10¹⁰ CFU·mL⁻¹ and 10¹¹ CFU·mL⁻¹ of said at least one probiotic bacterium.

Spray Drying

Spray drying may be performed according to any suitable known method and using any suitable known spray dryer of the art, as long as it comprises at least the following steps:
a) atomization, namely the transforming the feed into droplets may be performed by any one of the following techniques:
   pressure nozzle atomization, wherein the spray is achieved by forcing the fluid through an orifice;
   two-fluid nozzle atomization, wherein the spray is achieved by mixing the feed with a compressed gas;
   centrifugal atomization, wherein the spray is achieved by passing the feed through or across a rotating disk.
b) drying: a constant rate phase ensures rapid evaporation of the moisture from the surface of the particle and is followed by a falling rate period, during which the drying is controlled by diffusion of water to the surface of the particle.

In some embodiments, prior to the atomization step, the biomass composition may be homogenised, preferably with mild mechanical agitation, for a minimum of 10 min, preferably for a time ranging from 15 min to 30 min.

In some embodiments, upon drying, the powder may be separated from moist gas. This step may be achieved by removing the fine particles with cyclones, bag filters, precipitators or scrubbers.

In some embodiments, upon drying, the powder may undergo cooling and subsequent packaging.

In some preferred embodiments, the spray drying is performed by two-fluid nozzle atomization, preferably by utilizing a spray dryer commercially available from GEA Niro A/S (Denmark), accordingly to the manufacturer's recommendations.

In some embodiments, the biomass composition according to the invention is sprayed into a gas, preferably having an inlet temperature in the range from 100 to 200° C., in a spray dryer, whereas the spray dryer has an outlet temperature of at most 80° C.

In some embodiments, suitable gases include air, nitrogen, argon, helium, carbon dioxide, and mixtures thereof. In some preferred embodiments, the gas comprises air. In some particular embodiments, the gas comprises dehumidified air. Dehumidification may be performed by any suitable dehumidifier, preferably to achieve a humidity of the inlet air ranging from 0.5 g kg⁻¹ to 0.5 g·kg^{-l} of water in the air, preferably around 1 g·kg⁻¹.

In some embodiments of step b) of the method, the inlet temperature of spray drying is ranging from 120° C. to 200° C., preferably ranging from 140° C. to 180° C.

In some embodiments of step b) of the method, an inlet temperature of spray drying ranging from 120° C. to 200° C. includes an inlet temperature of 130° C., 140° C., 150° C., 160° C., 170° C. 180° C. and 190° C.

In some embodiments of step b) of the method, the outlet temperature of spray drying is ranging from 55° C. to 80° C., preferably ranging from 60° C. to 75° C.

In some embodiments of step b) of the method, an outlet temperature of spray drying ranging from 55° C. to 80° C. includes an outlet temperature of 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C. and 79° C.

Freeze Drying

Freeze drying may be performed according to any suitable known method in the art and using any suitable equipment of the art, as long as it comprises at least a step of sublimation, intended to remove most or all the water contained in the composition comprising the probiotic biomass from step a).

In practice, the freeze drying step may comprise a freezing step followed by a step of decrease of the pressure.

In some embodiments, the freezing step may be performed at a temperature comprised from −20° C. to −196° C., preferably from −50° C. to −80° C.

In some embodiments, the step of decrease of the pressure may be performed as to obtained a final pressure comprised from 1 Pa to 10,000 Pa, preferably from 10 Pa to 1,000 Pa, most preferably from 25 Pa to 100 Pa.

Probiotic Powder

In one aspect, the invention relates to a probiotic powder comprising at least one probiotic bacterium obtained by a method according to the instant invention.

Particle Size Distribution

In some embodiments, the probiotic powder may be characterized by the size distribution of the particles contained in said powder.

In practice, the particle size distribution of powders, in particular “D0.9”, “D0.5” and “D0.1” values, may be determined by well-known methods of the prior art such as sieve analysis, laser light scattering, photo-analysis or optical counting methods.

In some embodiments, laser light scattering is particularly preferred to determine the size distribution of the particles contained in the probiotic powder.

Within the scope of the invention, by “D0.9 particle size” is meant that the particle size distribution is such that at least 90% of the particles have a particle size diameter of less than the specified value.

Within the scope of the invention, by “D0.5 particle size” is meant that the particle size distribution is such that at least 50% of the particles have a particle size diameter of less than the specified value.

Within the scope of the invention, by “D0.1 particle size” is meant that the particle size distribution is such that at least 10% of the particles have a particle size diameter of less than the specified value.

Within the scope of the invention, the term “about” before a “specific value” defines a range from “the specific value minus 10% of the specific value” to “the specific value plus 10% of the specific value”. For example, “about 50” defines a range from 45 to 55.

In some embodiments, D0.9 particle size is less than about 100 μm, which includes D0.9 particle sizes less than about 90 μm, 80 μm, 70 μm, 60 μm, 55 μm, 50 μm, 45 μm, 40 μm, 38 μm, 36 μm, 34 μm, 32 μm, 30 μm, 28 μm, 26 μm, 24 μm, 22 μm and 20 μm.

In some embodiments, D0.9 particle size is ranging from about 25 μm to about 90 μm, which includes D0.9 particle sizes of about 30 μm, 35 μm, 40 μm, 45 μm, 50 μm, 55 μm, 60 μm, 65 μm, 70 μm, 75 μm, 80 μm and 85 μm.

In some embodiments, D0.5 particle size is ranging from about 8 μm to about 50 μm, which includes D0.5 particle sizes of about 9 μm, 10 μm, 11 μm, 12 μm, 13 μm, 14 μm, 15 μm, 16 μm, 17 μm, 18 μm, 19 μm, 20 μm, 21 μm, 22 μm, 23 μm, 24 μm, 25 μm, 26 μm, 27 μm, 28 μm, 29 μm, 30 μm, 31 μm, 32 μm, 33 μm, 34 μm, 35 μm, 36 μm, 37 μm, 38 μm, 39 μm, 40 μm, 41 μm, 42 μm, 43 μm, 44 μm, 45 μm, 46 μm, 47 μm, 48 μm and 49 μm.

In some embodiments, D0.1 particle size is less than about 10 μm, which includes D0.1 particle sizes of less than about 9.5 μm, 9 μm, 8.5 μm, 8 μm, 7.5 μm, 7 μm, 6.5 μm, 6 μm, 5.5 μm, 5 μm, 4.5 μm, 4 μm, 3.5 μm, 3 μm, 2.5 μm and 2 μm.

In some embodiments, D0.1 particle size is ranging from about 6 μm to about 9 μm, which includes D0.1 particle sizes of about 6.2 μm, 6.4 μm, 6.6 μm, 6.8 μm, 7.0 μm, 7.2 μm, 7.4 μm, 7.6 μm, 7.8 μm, 8.0 μm, 8.2 μm, 8.4 μm, 8.6 μm and 8.8 μm.

In some embodiments, the probiotic powder comprises particles having:
a D0.9 particle size of less than about 100 μm, and/or
a D0.5 particle size ranging from about 8 μm to about 50 μm, and/or
a D0.1 particle size of less than about 10 μm.

In some embodiments, the probiotic powder comprises particles having:
a D0.9 particle size ranging from about 25 μm to about 90 μm, and/or
a D0.5 particle size is ranging from about 8 μm to about 50 μm, and/or
a D0.1 particle size ranging from about 6 μm to about 9 μm.

In some embodiments, the size distribution of the particles is characterized by a ratio D0.5 (TS 15-35%)/D0.5 (TS≤5%)>1, wherein:
“D0.5(TS 15-35%)” represents a D0.5 particle size measured from a probiotic powder obtained from a probiotic biomass resulting from a culture of probiotics in a whey-containing nutrient medium having a total solid content ranging from 15% by weight to 35% by weight, based on the total weight of the said whey-containing nutrient medium, and
“D0.5(TS≤5%)” represents a D0.5 particle size measured from a probiotic powder obtained from a probiotic biomass resulting from a culture of probiotics in a whey-containing nutrient medium having a total solid content equal or below 5% by weight, based on the total weight of the said whey-containing nutrient medium.

In some embodiments, a ratio D0.5 (TS 15-35%)/D0.5 (TS≤5%)>1 includes a ratio equal or above about 1.1, 1.2, 1.3, 1.4, 1.5, 1.6, 1.7, 1.8, 1.9, 2.0, 2.2, 2.4, 2.6, 2.8, 3.0, 3.2, 3.4, 3.6, 3.8, 4.0, 4.5, 5.0, 5.5 and 6.0.

In some embodiments, the size distribution of the particles of the probiotic powder is characterized by a ratio D0.5 (TS 15-35%)/D0.5 (TS≤5%)>1.3.

In some embodiments, the “Span” parameter is used to characterize the size distribution of powders, and is calculated as follows:

Span=(D0.9−D0.1)/D0.5

wherein D0.1, D0.5 and D0.9 represent the above defined parameters.

In some embodiments, the size distribution of the particles of the probiotic powder is characterized by a Span ranging from 1.2 to 3.0, preferably ranging from 1.4 to 2.8.

In some embodiments, a Span ranging from 1.2 to 3.0 includes 1.3, 1.4, 1.5, 1.6, 1.7, 1.8, 1.9, 2.0, 2.1, 2.2, 2.3, 2.4, 2.5, 2.6, 2.7, 2.8 and 2.9.

In some embodiments, the size distribution of the particles is characterized by a ratio Span (TS 15-35%)/Span (TS≤5%)>1, wherein:
“Span(TS 15-35%)” represents a Span value calculated from a probiotic powder obtained from a probiotic biomass resulting from a culture of probiotics in a whey-containing nutrient medium having a total solid content ranging from 15 to 35 wt %, and
“Span(TS≤5%)” represents a Span value calculated from a probiotic powder obtained from a probiotic biomass resulting from a culture of probiotics in a whey-containing nutrient medium having a total solid content equal or below 5 wt %.

In some embodiments, a ratio Span (TS 15-35%)/Span (TS≤5%)>1 includes a ratio equal or above about 1.1, 1.2, 1.3, 1.4, 1.5, 1.6, 1.7, 1.8, 1.9, 2.0, 2.2, 2.4, 2.6, 2.8, 3.0, 3.2, 3.4, 3.6, 3.8, 4.0, 4.5, 5.0, 5.5 and 6.0.

In some embodiments, the size distribution of the particles is characterized by a ratio D0.5 (TS 15-35%)/D0.5 (TS≤5%)≥1.3 and/or a ratio Span (TS 15-35%)/Span (TS≤5%)≥1.3.

Viability

In some embodiments, the probiotic powder may be characterized by a number of viable cells (CFU, colony forming units) per gram (CFU·g⁻¹) of said powder.

In some embodiments, the probiotic powder comprises at least 10⁸ CFU·g⁻¹ of said at least one probiotic bacterium.

In some embodiments, 10⁸ CFU·g⁻¹ of said at least one probiotic bacterium includes 1.2×10⁸ CFU·g⁻¹, 1.4×10⁸ CFU·g⁻¹, 1.6×10⁸ CFU·g⁻¹, 1.8×10⁸ CFU·g⁻¹, 2.0×10⁸ CFU·g⁻¹, 2.2×10⁸ CFU·g⁻¹, 2.4×10⁸ CFU·g⁻¹, 2.6×10⁸ CFU·g⁻¹, 2.8×10⁸ CFU·g⁻¹, 3.0×10⁸ CFU·g⁻¹, 3.5×10⁸ CFU·g⁻¹, 4.0×10⁸ CFU·g⁻¹, 4.5×10⁸ CFU·g⁻¹, 5.0×10⁸ CFU·g⁻¹, 5.5×10⁸ CFU·g⁻¹, 6.0×10⁸ CFU·g⁻¹, 6.5×10⁸ CFU·g⁻¹, 7.0×10⁸ CFU·g⁻¹, 7.5×10⁸ CFU·g⁻¹, 8.0×10⁸ CFU·g⁻¹, 8.5×10⁸ CFU·g⁻¹, 9.0×10⁸ CFU·g⁻¹, 9.5×10⁸ CFU·g⁻¹, 10⁹ CFU·g⁻¹, 2×10⁹ CFU·g¹, 3.10⁹ CFU·g⁻¹, 4×10⁹ CFU·g⁻¹, 5×10⁹ CFU·g⁻¹, 6×10⁹ CFU·g⁻¹, 7×10⁹ CFU·g⁻¹, 8×10⁹ CFU·g⁻¹, 9×10⁹ CFU·g⁻¹, 10¹⁰ CFU·g⁻¹, 2×10¹⁰ CFU·g⁻¹, 3.10¹⁰ CFU·g⁻¹, 4×10¹⁰ CFU·g⁻¹, 5×10¹⁰ CFU·g⁻¹, 6×10¹⁰ CFU·g⁻¹, 7×10¹⁰ CFU·g⁻¹, 8×10¹⁰ CFU·g⁻¹, 9×10¹⁰ CFU·g⁻¹ and 10¹¹ CFU·g⁻¹ of said at least one probiotic bacterium.

In practice, the cell viability (CFU, colony forming units) is measured accordingly to the known methods from the state in the art. In practice, serial dilutions of a sample comprising live microorganisms are performed and plated onto an agar containing nutrient medium. CFU are counted from the applicable dilution(s).

Survival Before/after Spray Drying or Freeze Drying

In some other embodiments, the probiotic powder may be characterized by the survival of probiotics after spray drying or freeze drying.

The survival of probiotics after spray drying or freeze drying is calculated as follows:

Survival=Nd/N0×100%,

wherein Nd refers to the probiotic population (CFU·g⁻¹) in powders after spray drying or freeze drying, while the initial population N0 (CFU·g⁻¹) is calculated from N+ and the total solid content (TS) of medium as following:

N0=N+×(1−TS)/TS.

The log reduction of bacteria after 120 days storage is calculated as follows:

Log reduction=Log Nd−Log N120,

wherein the N120 means the bacteria population in the powders when storage for 120 days.

In some embodiments, the probiotic powder may be characterized by a ratio Survival (TS 15-35%)/Survival (TS≤5%)≥1.5, wherein:
“Survival (TS 15-35%)” represents a survival measured from a probiotic powder obtained from a probiotic biomass resulting from a culture of probiotics in a whey-containing nutrient medium having a total solid content ranging from 15 to 35 wt %, and
“Survival (TS≤5%)” represents a survival measured from a probiotic powder obtained from a probiotic biomass resulting from a culture of probiotics in a whey-containing nutrient medium having a total solid content equal or below 5 wt %.

In some embodiments, the ratio Survival (TS 15-35%)/Survival (TS≤5%) is ranging from 1.5 to 50, which includes 1.6, 1.7, 1.8, 1.9, 2.0, 2.2, 2.4, 2.6, 2.8, 3, 4, 5, 6, 7, 8, 9, 10, 11, 12, 13, 14, 15, 16, 17, 18, 19, 20, 22, 24, 26, 28, 30, 32, 34, 36, 38, 40, 42, 44, 46 and 48.

In some preferred embodiments, the ratio Survival (TS 15-35%)/Survival (TS≤5%) is ranging from 1.5 to 25.

In some embodiments, the probiotic powder may be characterized by a ratio Survival (TS 15-35%)/Survival (TS≤(5+10-30%)≥1.5, wherein:
“Survival (TS 15-35%)” represents a survival measured from a probiotic powder obtained from a probiotic biomass cultured in a whey-containing nutrient medium having a total solid content ranging from 15 to 35 wt %, and
“Survival (TS≤(5+10-30%)” represents a survival measured from a probiotic powder obtained from a probiotic biomass cultured in a whey-containing nutrient medium having a total solid content of 5 wt %, and which total solid content was adjusted to 15 wt % or 35 wt % with freshly added whey after the growth of said probiotics is achieved.

In some embodiments, the ratio Survival (TS 15-35%)/Survival (TS≤(5+10-30)%) is ranging from 1.5 to 10, which includes 1.6, 1.7, 1.8, 1.9, 2.0, 2.2, 2.4, 2.6, 2.8, 3, 4, 5, 6, 7, 8 and 9.

In some preferred embodiments, the ratio Survival (TS 15-35%)/Survival (TS≤(5+10-30)%) is ranging from 2 to 6.

Use

In another aspect, the invention relates to a probiotic powder according to the invention for use for improving health of a human or an animal body.

In some embodiments, the probiotic powder is intended to be provided for human consumption.

In some embodiments, the probiotic powder may be provided for pets, such as dogs, cats, mice, rats, guinea pigs; horse; for economically important livestock, such as poultry, cattle, sheep, lambs, goats, pigs, struthios and aquaculture animals. As used herein, poultry encompasses chickens, hens, geese, turkeys, and quails.

The invention also relates to the use of a whey-containing nutrient medium having a total solid content ranging from above 25% by weight to up to 35% by weight, based on the total weight of the said whey-containing nutrient medium, for culturing at least one probiotic bacterium, for the preparation of a probiotic powder comprising the said at least one probiotic bacterium.

In some embodiment, a probiotic powder may be administered by oral route.

These compositions can be provided in the form of dissolved solutions or suspensions, tablets, coated tablets, capsules, syrups and the like. These compositions are prepared according to the usual methods in the food industry. The active ingredient can be incorporated into excipients normally used in these compositions, such as aqueous or non-aqueous carriers, talc, arabic gum, lactose, starch, magnesium stearate, cocoa butter, fatty substances of animal or plant origin, paraffin derivatives, glycols, various wetting agents, dispersants or emulsifiers, or preserving agents.

For oral use of a probiotic powder according to the invention, the use of an ingestible support is preferred. The ingestible support may be of diverse nature depending on the type of composition under consideration.

In some embodiments, the probiotic powder may be incorporated into a food product or a diet supplement.

A diet supplement may be formulated as milk or milk-based fermented products, products based on fermented cereals, milk-based powders, food products of candy type, chocolate, cereals, tablets, gel capsules or lozenges, oral supplements in dry form and oral supplements in liquid form are especially suitable for use as food supports.

In some embodiments, the probiotic powder according to the invention may be used in the preparation of dairy food products, such a, e.g. milk products, fermented milk products, yogurt, cheese, quark, chocolate mousse, frozen fermented dairy desserts, sour cream, and ice cream.

In some embodiments, the probiotic powder according to the invention may be used in the preparation of non-dairy food products, such a, e.g. vegetable-based drinks, peanut milk, fruit juices, soy-based products, cereal-based products, meat-based products and the like.

In some embodiments, the probiotic powder according to the invention may be used in the preparation of vegetable-based feed, e.g. maize silage for animal feeding. Probiotics can be inoculated to the raw material and take part to the fermentation process.

In some other embodiments, the probiotic powder according to the invention may be used in the preparation of veterinary compositions to be externally applied to the animal body, such as veterinary compositions suitable for being sprayed on the animal body. According to these embodiments, the probiotic bacteria contained in the probiotic powder according to the invention are aimed at colonizing the animal's skin.

In some embodiments, the probiotic powder according to the invention may be used in the preparation of powders for prophylaxis against bacterial infections from environmental origin. Probiotics powder can be inoculated to the litter of farm animals (e.g. cattle litter in barns) or of other, colonize the litter and avoid thus colonization by undesirable microorganisms (e.g. bacteria responsible for environmental mastitis).

In still other embodiments, the probiotic powder according to the invention may be used in the preparation of veterinary compositions or compositions for human use to be orally administered by inhalation, e.g. intranasally. According to these embodiments, the probiotic bacteria contained in the probiotic powder according to the invention are aimed at colonizing the respiratory tract of the administered human individual or of the administered animal.

A probiotic powder according to the invention may moreover be formulated with excipients and components that are commonly used for such oral compositions or food supplements, as for example, fatty and/or aqueous components, humectants, thickeners, preserving agents, texture agents, taste agents and/or coating agents, antioxidants, preserving agents and dyes that are common in the food industry.

In some embodiments, a probiotic powder according to the invention may be administered to a human or non-human body once, twice or three times a day, for one, two, three, four, five or six days, or one, two, three or four weeks, or one, two, three, four, five, six, or more months.

The dosage regimen of administration will be adapted by the skilled man according to the usual parameters taken into account in the field for setting a regimen of administration, such as, for example, the weight, the size, the age and/or the gender of the body.

In some embodiments, the probiotic powder comprising at least 10⁹ CFU·g⁻¹ of at least one probiotic bacterium may be administered at a dose ranging from 0.01 mg to 10000 mg per day, preferably from 1 mg to 1000 mg per day.

In some embodiments, a dose ranging from 0.01 mg to 10000 mg per day includes a dose of 0.02 mg, 0.03 mg, 0.04 mg, 0.05 mg, 0.06 mg, 0.07 mg, 0.08 mg, 0.09 mg, 0.1 mg, 0.2 mg, 0.3 mg, 0.4 mg, 0.5 mg, 0.6 mg, 0.7 mg, 0.8 mg, 0.9 mg, 1 mg, 2 mg, 3 mg, 4 mg, 5 mg, 6 mg, 7 mg, 8 mg, 9 mg, 10 mg, 20 mg, 30 mg, 40 mg, 50 mg, 60 mg, 70 mg, 80 mg, 90 mg, 100 mg, 200 mg, 300 mg, 400 mg, 500 mg, 600 mg, 700 mg, 800 mg, 900 mg, 1000 mg, 2000 mg, 3000 mg, 4000 mg, 5000 mg, 6000 mg, 7000 mg, 8000 mg and 9000 mg per day.

In some embodiments, the probiotic powder comprising at least 10⁹ CFU·g⁻¹ of at least one probiotic bacterium, may be mixed in a liquid and administered at a dose ranging from 0.01 mg·L⁻¹ to 10000 mg·L⁻¹ per day, preferably from 1 mg·L⁻¹ to 1000 mg·L⁻¹ per day.

In some embodiments, a dose ranging from 0.01 mg·L⁻¹ to 10000 mg·L⁻¹ per day includes a dose of 0.02 mg·L⁻¹, 0.03 mg·L⁻¹, 0.04 mg·L⁻¹, 0.05 mg·L⁻¹, 0.06 mg·L⁻¹, 0.07 mg·L⁻¹, 0.08 mg·L⁻¹, 0.09 mg·L⁻¹, 0.1 mg·L⁻¹, 0.2 mg·L⁻¹, 0.3 mg·L⁻¹, 0.4 mg·L⁻¹, 0.5 mg·L⁻¹, 0.6 mg·L⁻¹, 0.7 mg·L⁻¹, 0.8 mg·L⁻¹, 0.9 mg·L⁻¹, 1 mg·L⁻¹, 2 mg·L⁻¹, 3 mg·L⁻¹, 4 mg·L⁻¹, 5 mg·L⁻¹, 6 mg·L⁻¹, 7 mg·L⁻¹, 8 mg·L⁻¹, 9 mg·L⁻¹, 10 mg·L⁻¹, 20 mg·L⁻¹, 30 mg·L⁻¹, 40 mg·L⁻¹, 50 mg·L⁻¹, 60 mg·L⁻¹, 70 mg·L⁻¹, 80 mg·L⁻¹, 90 mg·L⁻¹, 100 mg·L⁻¹, 200 mg·L⁻¹, 300 mg·L⁻¹, 400 mg·L⁻¹, 500 mg·L⁻¹, 600 mg·L⁻¹, 700 mg·L⁻¹, 800 mg·L⁻¹, 900 mg·L⁻¹, 1000 mg·L⁻¹, 2000 mg·L⁻¹, 3000 mg·L⁻¹, 4000 mg·L⁻¹, 5000 mg·L⁻¹, 6000 mg·L⁻¹, 7000 mg·L⁻¹, 8000 mg·L⁻¹ and 9000 mg·L⁻¹ per day.

As shown in the example section below, the use of a two-in-one nutrient medium according to the invention allows:
performing a two-step spray drying of a probiotic biomass composition comprising the steps of (i) growing a biomass of probiotics and (ii) spray drying said biomass composition in order to obtained a probiotic powder;
increasing the viability of the probiotics of said probiotic powder;
increasing the survival of said probiotics after spray drying;
increasing the viability and the survival of the probiotics from said probiotic powder after long term storage.

The present invention is further illustrated, without in any way being limited to, the examples below.

### EXAMPLES

### Example 1

### 1. Material and Methods

### 1.1. Strains and Culture Condition

The probiotic strain Lactobacillus casei BL23 was provided by UMR1219 MICALIS, (INRA-AgroParisTech, Jouy-En-Josas, France) and Propionibacterium freudenreichii ITG P20 was kept and pre-cultured by the CIRM-BIA Biological Resource Center (Centre International de Ressources Microbiennes-Bactéries d'Intérêt Alimentaire, INRA, Rennes, France). L. casei was activated by inoculating (1% inoculum size) in MRS Broth and cultivating statically at 37° C. for 16 h, while the P. freudenreichii was inoculated (1% inoculum size) in YEL broth and cultivated statically at 30° C. for 50 h.

### 1.2. Growth in Sweet Whey Media

The sweet whey powder (Lactalis ingredients, Mayenne, France) was used to prepare the sweet whey media with different TS in this work. The composition of sweet whey powder was analyzed by the procedures described by (Gaucher et al., 2008)(Table 1).

TABLE 1
Physical and chemical analysis of the sweet whey powder (the
composition is expressed as (w/w)%, i.e. weight of component/weight
of total solid content).

| | |
|---|---|
| Total solids | 94.81 |
| pH | 6.52 |
| Total nitrogen (%) | 11.81 |
| Non-protein nitrogen (%) | 3.11 |
| Lactose (%) | 67.91 |
| Ashes (%) | 6.85 |
| Calcium (%) | 0.37 |
| Magnesium (%) | 0.11 |
| Sodium (%) | 0.59 |
| Potassium (%) | 2.27 |
| Chloride (%) | 1.72 |
| Phosphate (%) | 1.14 |
| Citrate (%) | 2.26 |

The sweet whey powder was dissolved in deionized water to obtain media with the final total solids (TS, w/w) at 5%, 10%, 20%, 30% and 40% respectively. Additionally, the media with casein peptone were prepared by adding the casein peptone plus (Organotechnie, France) in the above sweet whey media at the concentration of 0.5% w/w (Cousin et al., 2012). These media were autoclaved at 100° C. for 30 min before inoculation of probiotic bacteria. The L. casei was inoculated at 1% inoculum size in the different sweet whey media (i.e. with different TS of sweet whey and with/without casein peptone) with the MRS preculture. The inoculated media were incubated statically at 37° C. for 48 h. Similarly, P. freudenreichii was inoculated to the sweet whey media from YEL preculture and incubated statically at 30° C. for 120 h (5 days).

### 1.3. Spray Drying

The probiotic culture grown in the casein peptone-supplemented media were used for spray drying. In addition to dry these bacteria culture directly, another group of samples were prepared by growing bacteria in 5% casein peptone-supplemented medium but increasing the TS value to 30% by adding the sweet whey powder before drying.

Spray drying was carried out with a pilot-scale Mobile Minor™ spray dryer (GEA Niro A/S, Denmark) with the maximal evaporation rate at 5 kg water h⁻¹. A two-fluid spray nozzle with the orifice diameter 0.8 mm was used in couple with a peristaltic pump (Watson-Marlow, France) for feeding and atomization. The humidity of inlet air was controlled at around 1 g·kg⁻¹ of water in the air by a dehumidifier (Munters, Sweden). Spray drying parameters were monitored by the SD²P® software (Schuck et al., 2009). All the sample-contacted parts in the dryer including the nozzle, chamber etc. were washed by hot water at 90° C. The 200° C. inlet temperature was used to dry the dryer and inactivate the possible microorganisms 2 h before drying the probiotic culture.

All the media (1 L) were agitated moderately 10 min before spray drying. Considering the intrinsic resistance of both strains, the inlet temperature of spray drying was set at 140° C. for L. casei (fragile strain) and 180° C. for P. freudenreichii (robust strain). The outlet temperature was 63±2° C. for L. casei and 73±2° C. for P. freudenreichii, and the outlet air relative humidity was controlled at 10±1% by adjusting the feeding rate. The water content and water activity of the powders were tested according to the methods described by (Schuck et al., 2012).

### 1.4. Size Distribution Measurement and Scanning Electron Microscopy

The size distribution of powders was measured by a laser light scattering with the MasterSizer 2000 equipped with a 5-mW helium-neon laser (Malvern Instruments, UK). The dry powder feeder attachment was used in couple with the standard optical model presentation for dispersion of powders in the air. The parameters D_{0.5} and Span were used to characterize the size distribution of powders, in which D_{0.5} means that 50% of the particles had diameters smaller than this criterion, while Span of powders was calculated as:

Span=(D_{0.9}−D_{0.1})/D_{0.5} (1)

wherein D_{0.9} and D_{0.1} mean the criterion of diameters which 90% and 10% of the particles were smaller than respectively.

The result was from the mean of 2 independent samplings which had 3 successive measurements each time.

The powders of L. casei from the media with 5% and 30% TS were fixed on the carbon tape and then sputter-coated with the gold-palladium. These powder samples were observed by a scanning electron microscopy (JSM 7100F, JOEL, USA) at 5 kV (Fu et al., 2013).

### 1.5. Storage

The powders were collected and sealed in the sterilized polystyrene bottles (Gosselin, France). The sample contained bottles were stored under a controlled temperature of 4° C. and kept away from light. The samples were analyzed in a 30 day interval for 120 days (4 months).

### 1.6. Enumeration and Quantification of Bacteria Viability

The number of viable cells (CFU) was firstly measured after growth (i.e. before spray drying). The bacteria culture after 10 min agitation was diluted serially (1 mL to 9 mL) in peptone water (0.1% w/v). The powder samples after spray drying or during storage were rehydrated by dissolving 1 g powder in 9 mL peptone water before serial dilutions. The diluted sample of L. casei was poured into MRS agar and incubated at 37° C. for 48 h (aerobic condition), while the dilution of P. freudenreichii was poured into YEL agar and incubated at 30° C. for 6 days under anaerobic condition (Anaerocult®, Merck KgaA, Germany).

The dependency of bacteria growth on the supplement of casein peptone was calculated as following equation:

Dependency=(N⁺−N⁻)/N⁻ (2)

wherein N⁺ means the bacteria population (CFU·mL⁻¹) in the medium with supplement of casein peptone, N⁻ means without supplement of casein peptone.

The survival of bacteria after spray drying was calculated as:

Survival=N_{d}/N₀×100% (3)

wherein N_{d} refers to the bacterial population (CFU·g⁻¹) in powders after spray drying, while the initial population N₀ (CFU·g⁻¹) was calculated from N⁺ and the total solid content (TS) of medium as following:

N₀=N⁺×(1−TS)/TS (4)

The log reduction of bacteria after 120 days storage was calculated as

Log reduction=Log N_{d}−Log N₁₂₀ (5)

wherein the N₁₂₀ means the bacteria population in the powders when storage for 120 days.

### 1.7. Statistical Analysis

All the experiments were repeated at least three times. The results were presented as mean value with standard error. Significant differences (p<0.05) between the mean values were determined by Tukey's test. The statistical analysis was carried out using R 3.2.1 with the package of ‘Rcmdr’ (the R Development Core Team).

### 2. Results and Discussion

### 2.1. Growth of Probiotics in Sweet Whey

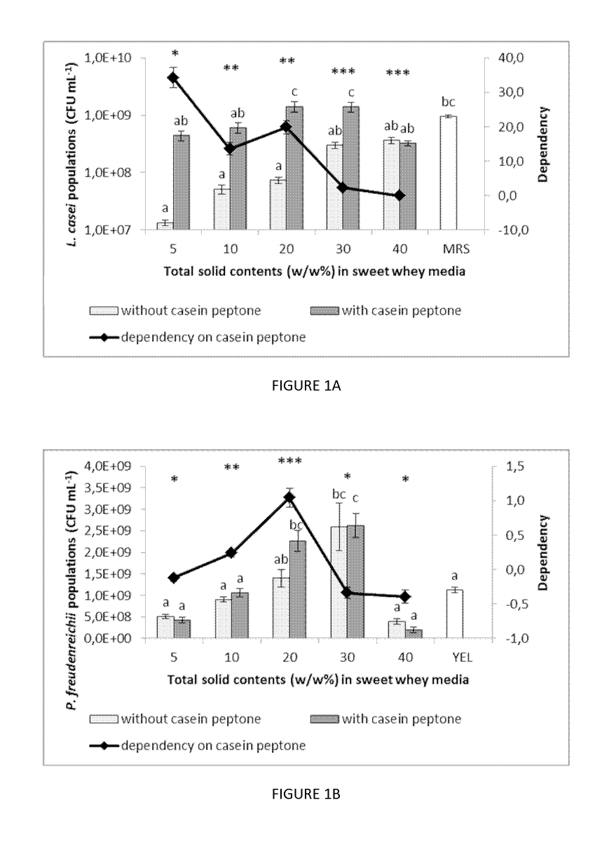

The growth of bacteria was compared in different nutrient media constituted by different TS of sweet whey, with or without supplement of casein peptone. As shown in the FIG. 1A, the final population of L. casei in sweet whey media without casein peptone increased as TS increased from 5% to 40%. Indeed, the supplement of casein peptone significantly enhanced the final L. casei population from the TS of 5% to 30%, but not for that of 40%. The dependency of L. casei growth on casein peptone decreased when improving the TS of sweet whey. In these casein peptone supplemented media, the final L. casei population approximately 2×10⁹ CFU·mL⁻¹ was reached for media with 20% and 30% TS, i.e. slightly higher than that obtained in MRS broth. The final population of P. freudenreichii also showed a similar trend when increasing the TS of sweet whey (FIG. 1B). Specifically, the population also increased as the TS increased from 5% to 30%, but with a large reduction at 40%. The optimal TS values were located ranging from 20% to 30% as well, with higher populations than that obtained in YEL broth (˜1.0×10⁹ CFU·mL⁻¹). The casein peptone supplemented medium with 30% TS produced the highest population of P. freudenreichii (˜2.5×10⁹ CFU·mL⁻¹) among all the tested media in this work. However, compared to L. casei, the growth of P. freudenreichii in sweet whey media was less dependent on casein peptone. The maximal improvement was obtained when adding casein peptone in sweet whey with 20% TS. However, this dependency was around 1.0, which means that only one time improvement of P. freudenreichii population was found after the supplement of casein peptone.

The results indicate that increasing the TS of sweet whey to the range between 20% and 30% was able to improve the biomass production of both probiotic strains. As mentioned before, these higher TS values will be beneficial for the following spray drying. The improvement in the final bacteria populations may be caused by the richer nutrients in the media with higher TS value. Although the osmotic pressure was also higher in these media, the time of growth may be long enough to trigger the stress response of these two strains to adapt the high-osmolality environments (Wood, 2011). For example, it has been reported that both of these two strains were able to accumulate the intracellular polyphosphate which relates to the improvement of bacteria stress tolerance (Alcántara et al., 2014; Thierry et al., 2011). The presence of large amount of phosphate in the media with 20% or 30% TS may facilitate the accumulation of polyphosphate by uptake of phosphate from extracellular environment. However, when increasing the TS of sweet whey to 40%, the inhibition effect caused by the hyper osmolality and energy consumption in osmoregulation started to be deleterious for growth of the two strains.

### 2.2. Spray Drying of Probiotic Cultured in Sweet Whey

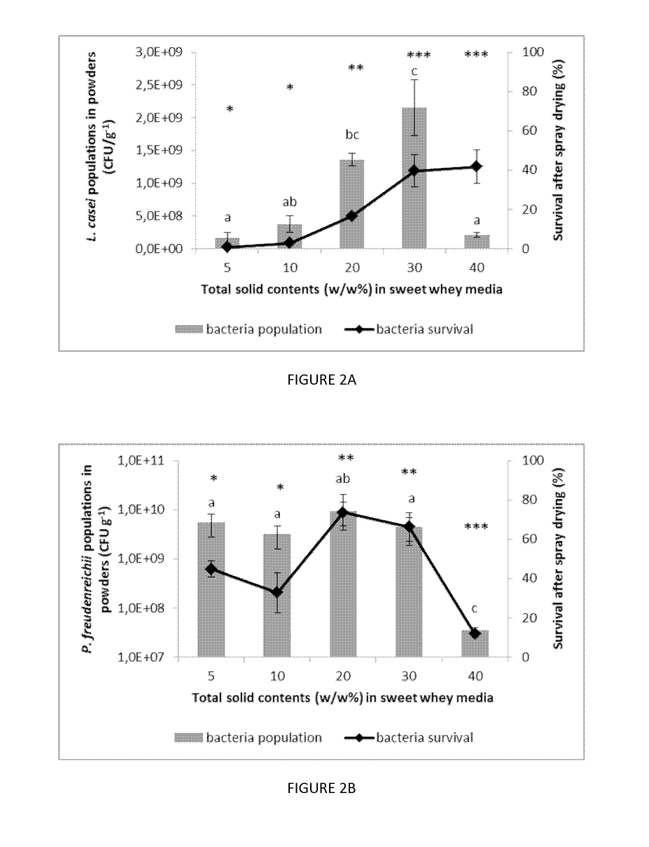

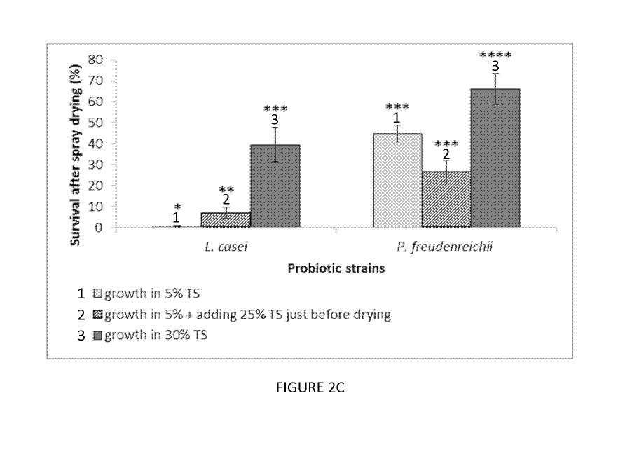

The remaining viability of probiotics was expressed as the population and survival of bacteria after spray drying. The water content and water activity values of all powders were 6±1% and 0.2±0.05% respectively. As shown in FIG. 2A, the survival of L. casei in powders after spray drying increased as the TS increased from 5% to 40%, with a maximal survival at around 40% for the TS values at 30% and 40%. In comparison with the powder from the medium of 5% TS, the survival in the powder from the medium of 30% TS was improved by approximately 60 folds (˜0.6% survival for 5% TS and ˜40% survival for 30% TS). Correspondingly, the population of L. casei in powders also increased as the TS increased from 5% to 30%, but with a reduction at that of 40% TS due to the relatively low population of L. casei in the medium of 40% TS before drying. For the strain of P. freudenreichii, the maximal survival (i.e. 70%) after spray drying appeared at the media with 20% and 30% TS. Similarly with that of L. casei, the media with low TS resulted in a lower survival at around 40% after spray drying. The population of viable bacteria in the powders from media with 5%, 10%, 20% and 30% were all higher than the level of 10⁹ CFU·g⁻¹. The highest population was found in the medium of 20% TS, which reached the level of 10¹⁰ CFU·g⁻¹. The P. freudenreichii growing in medium with 40% TS showed the lowest survival and remaining population after spray drying.

When comparing between two probiotic strains, the survival of P. freudenreichii was generally higher than that of L. casei after spray drying, albeit the higher drying temperature being used. It implied that the P. freudenreichii strain was more tolerated during spray drying than the L. casei strain. It is known that P. freudenreichii is general heat resistant species which were often used for making Emmental-type cheeses in which the curd is heated at 50° C.˜55° C. (Fox et al., 2004; Thierry et al., 2011). It has been described that P. freudenreichii accumulates the intracellular trehalose and glycogen in addition to polyphosphate, which involve in improving the bacterial tolerance against heat and/or desiccated stresses (Boyaval et al., 1999; Falentin et al., 2010).

To consider both the survival and final viable population of the probiotics in powders, the optimal TS value of sweet whey media was located between 20% and 30%. In this range, the two strains also presented the optimal biomass production. The improvement of survival may be caused by the robust cellular tolerance induced by higher osmolality in the media with TS from 20% to 30%. However, it could also be due to the higher dry matter (i.e. TS values). The relatively fine powders would be formed from the media with low TS due to the lower viscosity and less solid content within the particles, which may result in the longer retention time within the dryer (Jeantet et al., 2008). Besides, the lower solid content in medium also indicates that the less amount of wall material can be used to encapsulate the bacteria cells. In other words, the bacteria in the medium with lower TS may be exposed to the hot air to a larger extent (Perdana et al., 2014). Therefore, the lower solid content in the feeds seems to be disadvantageous for production powders with live probiotic bacteria.

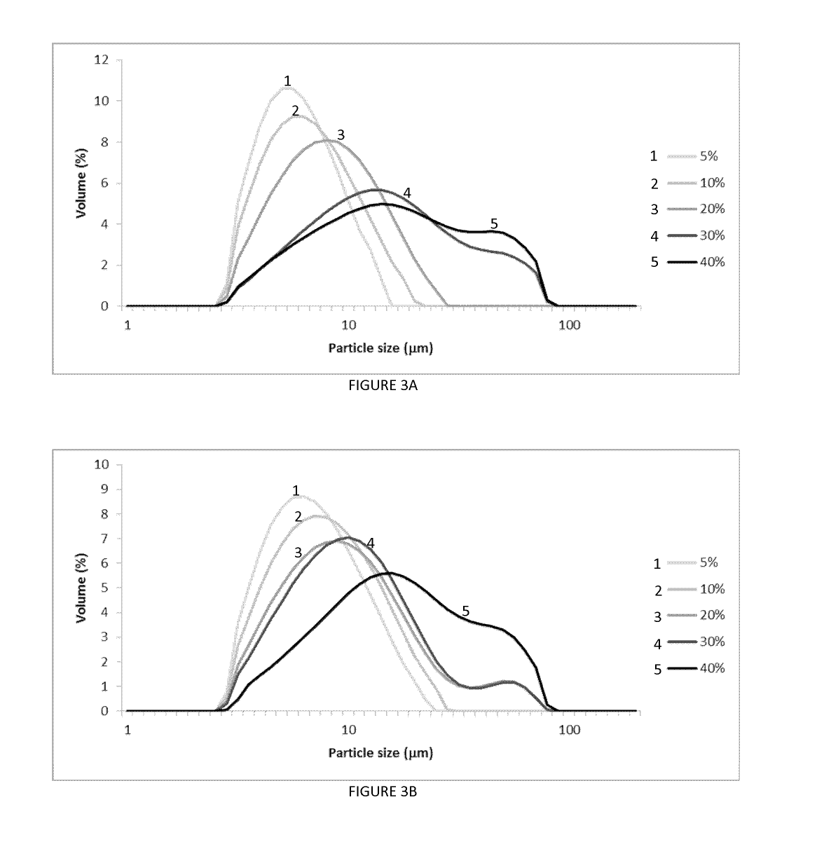

In order to investigate the contribution of increased TS on the improved survival of probiotics after spray drying, a drying experiment was conducted by growing bacteria in 5% TS medium but increasing the TS to 30% immediately before drying (FIG. 3C). After increasing the TS, the survival of L. casei after spray drying was improved to around 6% in comparison with that of growing and drying at 5% TS. However, it is still significantly lower than that of growing and drying at 30% TS. In contrast, the survival of P. freudenreichii decreased slightly (from 45% to 26%) when improving TS from 5% to 30% before drying, while growth in 30% TS significantly enhanced the spray drying survival to 70%.

The results indicated that the improvement of the probiotic survival after spray drying was not caused or only caused by the higher TS. The enhanced bacterial intrinsic tolerance triggered by high osmolality during growth may play the decisive role in the remaining of higher survival after spray drying. It has been reported that the salt-adaptation of Lactobacillus paracasei strain with high osmolality before spray drying (by 0.3 M NaCl) could improve the bacteria survival after heat treatment and spray drying (Desmond et al., 2001). In this work, the two bacteria strains were exposed to the high osmolality during the growth time (2 days for L. casei and 5 days for P. freudenreichii). It is known that reaching cellular homeostasis by accumulation of compatible solutes is an energy-dependent bacterial osmoregulation process (Guchte et al., 2002; Wood, 2011). In other words, the induction time is required for bacteria to adapt the adverse environments or trigger the stress response. In contrast to the bacteria growing in normal osmolality but exposed to the osmolality before drying, growing bacteria in the medium with high osmolality may have more enough time to accumulate the intracellular compatible solutes either by synthesis per se or uptake from environment (FIG. 3).

### 2.3. Size Distribution and Morphology of Powders

The size distribution of powders is shown in FIG. 3 and Table 2.

TABLE 2
The size distribution of spray dried powders from media with different TS

| | | | | |
|---|---|---|---|---|
| 5% | 10% | 20% | 30% | 40% |

L. casei

| | | | | | |
|---|---|---|---|---|---|
| D_{0.5} | 6.1 ± 0.1 | 6.9 ± 0.1 | 8.5 ± 0.1 | 15.3 ± 0.5 | 17.1 ± 0.1 |
| (μm) | | | | | |
| Span | 1.1 ± 0.1 | 1.3 ± 0.1 | 1.5 ± 0.1 | 2.8 ± 0.1 | 2.8 ± 0.1 |

P. freudenreichii

| | | | | | |
|---|---|---|---|---|---|
| D_{0.5} | 7.2 ± 0.1 | 8.2 ± 0.1 | 10.0 ± 0.1 | 10.7 ± 0.1 | 17.6 ± 0.1 |
| (μm) | | | | | |
| Span | 1.4 ± 0.1 | 1.5 ± 0.1 | 2.2 ± 0.1 | 2.0 ± 0.1 | 2.6 ± 0.1 |

For both strains, the size distribution of powders right shifted as the increasing of TS in the media. This result also corresponded to the observation by scanning electron microscopy. The powders dried from the media with high TS at 30% showed the larger span of size distribution (Span >2), but with the D_{0.5} larger than 10 μm. The powders from 5% medium were finer, with the narrow size distribution (Span between 1 and 1.5) peaking below 10 μm. However, it was difficult to find the bacteria cells on the surface of powders in the SEM observation, even for the powders obtained from the medium with 5% TS. It indicated that the sweet whey with the 5% TS was already sufficient as the wall materials for microencapsulation of bacteria in spray drying. It coincided with the observation of spray dried powders of Lactobacillus plantarum with trehalose as drying medium (Perdana et al., 2014).

### 2.4. Viability of Probiotics During Storage

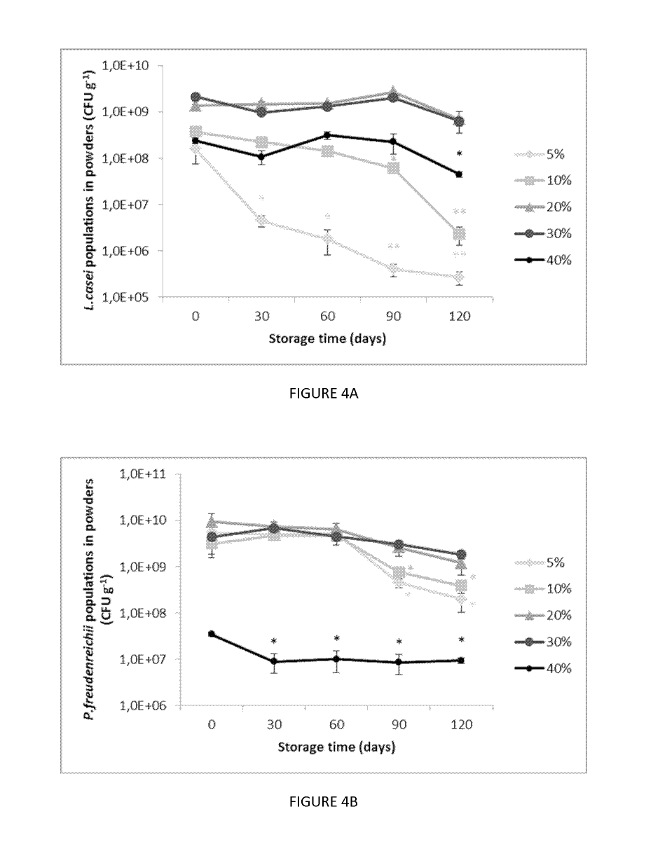

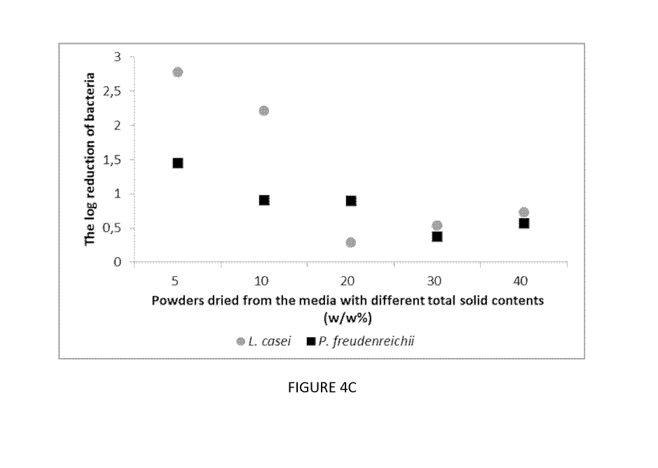

The changes of bacteria population in powders were monitored during storage at 4° C. for 120 days (FIG. 4). For both L. casei and P. freudenreichii strains, the viabilities in the powders dried from the media with TS 20%˜40% remained constant with a maximal 0.89 log reduction. After storage for 120 days, L. casei population in the powders from 20% and 30% media remained the highest population at around 7×10⁸ CFU·g⁻¹, while the P. freudenreichii at around 2×10⁹ CFU·g⁻¹. Since the initial bacteria populations in the powders from 40% medium were relatively low, the L. casei and P. freudenreichii populations were at around 4×10⁷ and 1×10⁷ CFU·g⁻¹ respectively after 120 days. In comparison, the viable L. casei population in the powder from 5% medium significantly dropped during the entire storage time, with a final population below 10⁶ CFU·g⁻¹ at the end of storage (˜2.8 log reduction at 120 days). Meanwhile, the L. casei in the powder from 10% medium remained unchanged during the first 90 days (viability loss <1 log), but displayed a large reduction after 90 days (˜2.2 log reduction at 120 days). The powders of P. freudenreichii from the 5% and 10% media exhibited a considerably steady viability during the first 60 days. Although there was reduction of viability after storage afterward (˜1.5 and 1 log reduction for 5% and 10% respectively), the final viable bacteria population still retained above 10⁸ CFU·g⁻¹ at the end of 120 days. The ability of P. freudenreichii to accumulate polyphosphate, trehalose and glycogen as energy and carbon storage compounds, as well as compatible solutes, could allow better survival throughout the long-term storage (Boyaval et al., 1999; Cardoso et al., 2007; Thierry et al., 2011).

### Example 2

### 1. Material and Methods

### 1.1. Strains and Growth in Sweet Whey Media

The probiotic bacteria used herein were provided by the Centre International de Ressources Microbiennes-Bactéries d'Intérêt Alimentaire (CIRM-BIA, Rennes, France). They are depicted in Table 3 below:

| Strain | Source |
|---|---|
| Bifidobacterium longum | CIRM-BIA 1336 |
| Lactobacillus acidophilus | CIRM-BIA 1674 |
| Lactobacillus delbrueckii subsp. bulgaricus | CIRM-BIA 1666 |
| Lactobacillus plantarum | CIRM-BIA 466 |
| Lactobacillus reuteri | CIRM-BIA 522 |
| Lactobacillus rhamnosus | CIRM-BIA 607 |

The probiotic lactobacilli strain were cultured in MRS medium, while the bifidobacteria strain was culture in MRS supplemented with 0.05% cysteine for 16 h (B. longum and L. acidophilus at 37° C., L. bulgaricus at 43° C., and other lactobacilli strains at 30° C.).

The sweet whey powder (Lactalis ingredients) was dissolved in deionized water to obtain media with the final total solids (TS, w/w) at 5 wt % and 30 wt %, respectively. These media were autoclaved at 115° C. for 15 min before inoculation of probiotic bacteria. The probiotic bacteria were inoculated at 4% inoculum size in the different sweet whey media or in MRS medium with the MRS preculture. The inoculated media were incubated statically at 30° C., 37° C. or 43° C. for 24 h (corresponding to the growth temperature above mentioned).

### 1.2. Heat Treatment

Following the growth in sweet whey medium, the biomass composition is brought to a temperature of 60° C. for 10 min. CFU·mL⁻¹ are measured prior and after the heat treatment as to determine the survival of the probiotic bacteria.

### 2. Results and Discussion

TABLE 4
Population of bacteria (data presented as Log₁₀ CFU · mL⁻¹) after growth
in different sweet whey-containing media (4% inoculum, 24 h culture)

| | | | L. delbrueckii | | | |
|---|---|---|---|---|---|---|
| | | | subsp. | | | |
| | B. longum | L. acidophilus | bulgaricus | L. plantarum | L. reuteri | L. rhamnosus |
| MRS | 9.44 | 9.08 ± 0.18 | 8.26 | 9.81 | 8.46 ± 0.23 | 9.59 ± 0.01 |
| 5% | 8.54 ± 0.10 | 8.00 ± 0.06 | 8.00 ± 0.06 | 8.61 ± 0.19 | 8.32 ± 0.03 | 9.24 ± 0.01 |
| whey¹ | | | | | | |
| 30% | 6.00 | 8.00 ± 0.06 | 7.65 ± 0.49 | 9.00 ± 0.21 | 8.07 ± 0.10 | 9.31 ± 0.10 |
| whey¹ | | | | | | |

¹Sweet whey.

As can be seen from Table 4, Bifidobacterium longum and all Lactobacillus species that are assayed substantially grow in sweet whey-containing nutrient medium, at 5 wt % and 30 wt %.

TABLE 5
Survival of bacteria (data presented as Log₁₀ N/N₀) after heat treatment

| | L. acidophilus | L. plantarum | L. reuteri | L. rhamnosus |
|---|---|---|---|---|
| MRS | −4.75 ± 0.18 | <−5.81 | <−4.49 | −4.85 ± 0.06 |
| 5% whey¹ | <−4.00 | <−4.63 | −0.79 ± 0.03 | <−5.24 |
| 30% | −3.01 ± 0.01 | −4.00 ± 0.03 | −0.56 ± 0.10 | −2.81 ± 0.01 |
| whey¹ | | | | |

¹Sweet whey.

After heat treatment, namely 60° C. for 10 min, the sweet whey-containing nutrient medium at 30 wt % significantly allows an increased protection of the probiotic bacteria, as compared to the sweet whey-containing nutrient medium at 5 wt %. The heat treatment performed herein represents harsher conditions than the spray drying technique. Indeed, although the temperatures of the heat treatment are similar to the temperatures performed in the spray drying, the heat treatment induces an overall heat stress that is superior to the overall heat stress encountered in spray drying. Therefore, the resistance of the bacteria to the heat treatment indicates that these bacteria would eventually display a higher survival at the end of a spray drying step, as disclosed in the present description.

### REFERENCES

Alcántara, C., Blasco, A., Zúĩga, M., & Monedero, V. (2014). Accumulation of Polyphosphate in Lactobacillus spp. and Its Involvement in Stress Resistance. Applied and Environmental Microbiology, 80(5), 1650-1659.
Ananta, E., Volkert, M., & Knorr, D. (2005). Cellular injuries and storage stability of spray-dried Lactobacillus rhamnosus GG. International Dairy Journal, 15(4), 399-409.
Barbosa, J., Borges, S., & Teixeira, P. (2015). Influence of sub-lethal stresses on the survival of lactic acid bacteria after spray-drying in orange juice. Food Microbiology, 52, 77-83.
Boyaval, P., Deborde, C., Corre, C., Blanco, C., & Bégué, É. (1999). Stress and osmoprotection in propionibacteria. Le Lait, 79(1), 59-69.
Cardoso, F. S., Castro, R. F., Borges, N., & Santos, H. (2007). Biochemical and genetic characterization of the pathways for trehalose metabolism in Propionibacterium freudenreichii, and their role in stress response. Microbiology, 153(1), 270-280.
Chirife, J., Herszage, L., Joseph, A., & Kohn, E. S. (1983). In vitro study of bacterial growth inhibition in concentrated sugar solutions: microbiological basis for the use of sugar in treating infected wounds. Antimicrobial Agents and Chemotherapy, 23(5), 766-773.
Cook, M. T., Tzortzis, G., Charalampopoulos, D., & Khutoryanskiy, V. V. (2012). Microencapsulation of probiotics for gastrointestinal delivery. Journal of Controlled Release, 162(1), 56-67.
Cousin, F. J., Louesdon, S., Maillard, M.-B., Parayre, S., Falentin, H., Deutsch, S.-M., Jan, G. (2012). The first dairy product exclusively fermented by Propionibacterium freudenreichii: A new vector to study probiotic potentialities in vivo. Food Microbiology, 32(1), 135-146.
Cousin, F. J., Mater, D. D. G., Foligné, B., & Jan, G. (2010). Dairy propionibacteria as human probiotics: A review of recent evidence. Dairy Science & Technology, 91(1), 1-26.
De Man, J. C., Rogosa, M., & Sharpe, M. E. (1960). A Medium for the Cultivation of Lactobacilli. Journal of Applied Bacteriology, 23(1), 130-135.
Desmond, C., Stanton, C., Fitzgerald, G. F., Collins, K., & Paul Ross, R. (2001). Environmental adaptation of probiotic lactobacilli towards improvement of performance during spray drying. International Dairy Journal, 11(10), 801-808.
Falentin, H., Deutsch, S.-M., Jan, G., Loux, V., Thierry, A., Parayre, S., Lortal, S. (2010). The Complete Genome of Propionibacterium freudenreichii CIRM-BIA1T, a Hardy Actinobacterium with Food and Probiotic Applications. PLoS ONE, 5(7), e11748. FAO/WHO. (2001). Health and Nutritional Properties of Probiotics in Food including Powder Milk with Live Lactic Acid Bacteria (pp. 1-34).
Foligné, B., Breton, J., Mater, D., & Jan, G. (2013). Tracking the microbiome functionality: focus on Propionibacterium species. Gut, gutjnl-2012-304393.
Foligné, B., Deutsch, S.-M., Breton, J., Cousin, F. J., Dewulf, J., Samson, M., Jan, G. (2010). Promising Immunomodulatory Effects of Selected Strains of Dairy Propionibacteria as Evidenced In Vitro and In Vivo. Applied and Environmental Microbiology, 76(24), 8259-8264.
Fox, P. F., McSweeney, P. L. H., Cogan, T. M., & Guinee, T. P. (2004). Cheese: Chemistry, Physics and Microbiology: Major Cheese Groups. Academic Press.
Fu, N., & Chen, X. D. (2011). Towards a maximal cell survival in convective thermal drying processes. Food Research International, 44(5), 1127-1149.
Fu, N., Woo, M. W., Selomulya, C., & Chen, X. D. (2013). Inactivation of Lactococcus lactis ssp. cremoris cells in a droplet during convective drying. Biochemical Engineering Journal, 79, 46-56.
Fu, W. Y., & Etzel, M. R. (1995). Spray Drying of Lactococcus lactis ssp. lactis C2 and Cellular Injury. Journal of Food Science, 60(1), 195-200.
Gaucher, I., Boubellouta, T., Beaucher, E., Piot, M., Gaucheron, F., & Dufour, E. (2008). Investigation of the effects of season, milking region, sterilisation process and storage conditions on milk and UHT milk physico-chemical characteristics: a multidimensional statistical approach. Dairy Science and Technology, 88(3), 291-312.
Golowczyc, M. A., Gerez, C. L., Silva, J., Abraham, A. G., Antoni, G. L. D., & Teixeira, P. (2010). Survival of spray-dried Lactobacillus kefir is affected by different protectants and storage conditions. Biotechnology Letters, 33(4), 681-686.
Guchte, M. van de, Serror, P., Chervaux, C., Smokvina, T., Ehrlich, S. D., & Maguin, E. (2002). Stress responses in lactic acid bacteria. Antonie van Leeuwenhoek, 82(1-4), 187-216.
Holzapfel, W. (2014). Advances in Fermented Foods and Beverages: Improving Quality, Technologies and Health Benefits. Elsevier.
Jantzen M, Göpel A, Beermann C. (2013). Direct spray drying and microencapsulation of probiotic Lactobacillus reuteri from slurry fermentation with whey. J Appl Microbiol. October; 115(4):1029-36.
Jeantet, R., Ducept, F., Dolivet, A., Méjean, S., & Schuck, P. (2008). Residence time distribution: a tool to improve spray-drying control. Dairy Science and Technology, 88(1), 31-43.
Kamada, N., Seo, S.-U., Chen, G. Y., & Núñez, G. (2013). Role of the gut microbiota in immunity and inflammatory disease. Nature Reviews Immunology, 13(5), 321-335.
Lavari, L., Páez, R., Cuatrin, A., Reinheimer, J., & Vinderola, G. (2014). Use of cheese whey for biomass production and spray drying of probiotic lactobacilli. Journal of Dairy Research, 81(03), 267-274.
Lian, W. C., Hsiao, H. C., & Chou, C. C. (2002). Survival of bifidobacteria after spray-drying. International Journal of Food Microbiology, 74(1-2), 79-86.
Lievense, L. C., & van't Riet, K. (1993). Convective Drying of Bacteria I. The Drying Processes. Advances in Biochemical Engineering, Biotechnology, 50, 45-63.
Lievense, L. C., & van't Riet, K. (1994). Convective Drying of Bacteria II. Factors Influencing Survival. Advances in Biochemical Engineering, Biotechnology, 51, 71-89.
Liu, H., Gong, J., Chabot, D., Miller, S. S., Cui, S. W., Ma, J., Wang, Q. (2015). Protection of heat-sensitive probiotic bacteria during spray-drying by sodium caseinate stabilized fat particles. Food Hydrocolloids, 51, 459-467.
Malik, A. C., Reinbold, G. W., & Vedamuthu, E. R. (1968). An evaluation of the taxonomy of Propionibacterium. Canadian Journal of Microbiology, 14(11), 1185-1191.
Paéz, R., Lavari, L., Vinderola, G., Audero, G., Cuatrin, A., Zaritzky, N., & Reinheimer, J. (2012). Effect of heat treatment and spray drying on lactobacilli viability and resistance to simulated gastrointestinal digestion. Food Research International, 48(2), 748-754.
Peighambardoust, S. H., Golshan Tafti, A., & Hesari, J. (2011). Application of spray drying for preservation of lactic acid starter cultures: a review. Trends in Food Science & Technology, 22(5), 215-224.
Perdana, J., Bereschenko, L., Fox, M. B., Kuperus, J. H., Kleerebezem, M., Boom, R. M., & Schutyser, M. A. I. (2013). Dehydration and thermal inactivation of Lactobacillus plantarum WCFS1: Comparing single droplet drying to spray and freeze drying. Food Research International, 54(2), 1351-1359.
Perdana, J., Fox, M. B., Siwei, C., Boom, R. M., & Schutyser, M. A. I. (2014). Interactions between formulation and spray drying conditions related to survival of Lactobacillus plantarum WCFS1. Food Research International, 56, 9-17.
Pessione, E. (2014). Interactive Probiotics. CRC Press.
Rodgers, S. (2008). Novel applications of live bacteria in food services: probiotics and protective cultures. Trends in Food Science & Technology, 19(4), 188-197.
Santivarangkna, C., Kulozik, U., & Foerst, P. (2007). Alternative drying processes for the industrial preservation of lactic acid starter cultures. Biotechnology Progress, 23(2), 302-315.
Schuck, P., Dolivet, A., & Jeantet, R. (2012). Analytical Methods for Food and Dairy Powders: Schuck/Analytical Methods for Food and Dairy Powders. Oxford, UK: Wiley-Blackwell.
Schuck, P., Dolivet, A., Méjean, S., Hervé, C., & Jeantet, R. (2013). Spray drying of dairy bacteria: New opportunities to improve the viability of bacteria powders. International Dairy Journal, 31(1), 12-17.
Schuck, P., Dolivet, A., Méjean, S., Zhu, P., Blanchard, E., & Jeantet, R. (2009). Drying by desorption: A tool to determine spray drying parameters. Journal of Food Engineering, 94(2), 199-204.
Semyonov, D., Ramon, O., Kaplun, Z., Levin-Brener, L., Gurevich, N., & Shimoni, E. (2010). Microencapsulation of Lactobacillus paracasei by spray freeze drying. Food Research International, 43(1), 193-202.
Stoklosa, A. M., Lipasek, R. A., Taylor, L. S., & Mauer, L. J. (2012). Effects of storage conditions, formulation, and particle size on moisture sorption and flowability of powders: A study of deliquescent ingredient blends. Food Research International, 49(2), 783-791.
Sunny-Roberts, E. O., & Knorr, D. (2009). The protective effect of monosodium glutamate on survival of Lactobacillus rhamnosus GG and Lactobacillus rhamnosus E-97800 (E800) strains during spray-drying and storage in trehalose-containing powders. International Dairy Journal, 19(4), 209-214.
Thierry, A., Deutsch, S.-M., Falentin, H., Dalmasso, M., Cousin, F. J., & Jan, G. (2011). New insights into physiology and metabolism of Propionibacterium freudenreichii. International Journal of Food Microbiology, 149(1), 19-27.
Vasseur, C., Baverel, L., Hébraud, M., & Labadie, J. (1999). Effect of osmotic, alkaline, acid or thermal stresses on the growth and inhibition of Listeria monocytogenes. Journal of Applied Microbiology, 86(3), 469-476.
Walter, R. P., Morris, J. G., & Kell, D. B. (1987). The Roles of Osmotic Stress and Water Activity in the Inhibition of the Growth, Glycolysis and Glucose Phosphotransferase System of Clostridium pasteurianum. Microbiology, 133(2), 259-266.
Wood, J. M. (2011). Bacterial Osmoregulation: A Paradigm for the Study of Cellular Homeostasis. Annual Review of Microbiology, 65(1), 215-238.
Wu, W. D., Liu, W., Gengenbach, T., Woo, M. W., Selomulya, C., Chen, X. D., & Weeks, M. (2014). Towards spray drying of high solids dairy liquid: Effects of feed solid content on particle structure and functionality. Journal of Food Engineering, 123, 130-135.
Yonekura, L., Sun, H., Soukoulis, C., & Fisk, I. (2014). Microencapsulation of Lactobacillus acidophilus NCIMB 701748 in matrices containing soluble fibre by spray drying: Technological characterization, storage stability and survival after in vitro digestion. Journal of Functional Foods, 6, 205-214.

1. A method for preparing a probiotic powder comprising at least one probiotic bacterium, said method comprising:
a) providing a probiotic biomass comprising at least one probiotic bacterium by culturing the at least one probiotic bacterium in a whey-containing nutrient medium having a total solid content ranging from above 25% by weight to up to 35% by weight, based on a total weight of the whey-containing nutrient medium; and
b) spray drying or freeze drying the probiotic biomass provided at step a) so as to obtain the probiotic powder.

2. The method according to claim 1, wherein said at least one probiotic bacterium comprises a probiotic bacterium selected from the group consisting of Bifidobacterium sp., Lactobacillus sp., Lactococcus sp., Propionibacterium sp., Streptococcus sp. and a mixture thereof.

3. The method according to claim 1, wherein said at least one probiotic bacterium comprises at least one probiotic bacterium selected from the group consisting of Bifidobacterium adolescentis, B. animalis, B. bifidum, B. breve, B. infantis, B. lactis, B. longum, Lactobacillus acidophilus, L. casei, L. delbrueckii, L. gasseri, L. paracasei, L. plantarum, L. reuteri, L. salivarius, L. rhamnosus, Lactococcus lactis, Propionibacterium freidenreichii, Streptococcus thermophilus and a mixture thereof.

4. The method according claim 1, wherein the whey-containing nutrient medium has a total solid content ranging from 26% by weight to 33% by weight, based on the total weight of the whey-containing nutrient medium.

5. The method according to claim 1, wherein, at step b), the inlet temperature of spray drying ranges from 120° C. to 200° C.

6. The method according to claim 1, wherein, at step b), the outlet temperature of spray drying ranges from 55° C. to 80° C.

7. The method according to claim 1, wherein said nutrient medium further comprises casein peptone as a nitrogen source.

8. A probiotic powder comprising at least one probiotic bacterium, which probiotic powder is obtained by
a) providing a probiotic biomass composition comprising at least one probiotic bacterium resulting from the culture of said probiotic bacterium in a whey-containing nutrient medium having a total solid content ranging from above 25% by weight to up to 35% by weight, based on the total weight of the whey-containing nutrient medium; and
b) spray drying or freeze drying the probiotic biomass provided at step a) so as to obtain the probiotic powder.

9. The probiotic powder according to claim 8, wherein a particle size distribution of the probiotic powder is characterized by a ratio D0.5
(TS15-35%)/D0.5(TS≤5%)>1, wherein:
D0.5(TS 15-35%) represents a D0.5 particle size measured from a probiotic powder obtained from a probiotic biomass resulting from a culture of probiotics in a whey-containing nutrient medium having a total solid content ranging from 15% by weight to 35% by weight, based on the total weight of the said whey-containing nutrient medium, and
D0.5(TS≤5%) represents a D0.5 particle size measured from a probiotic powder obtained from a probiotic biomass resulting from a culture of probiotics in a whey-containing nutrient medium having a total solid content equal or below 5% by weight, based on the total weight of the said whey-containing nutrient medium.

10. The probiotic powder according to claim 9, wherein the particle size distribution is characterized by a ratio D0.5 (TS 15-35%)/D0.5 (TS≤5%)>1.3.

11. The probiotic powder according to claim 10, wherein it comprises at least 10⁸ CFU·g⁻¹ of the at least one probiotic bacterium.

12. A method for improving health of a human or an animal, comprising
administering to the human or animal a quantity of the probiotic powder of claim 8 to improve the health of the human or the animal.

13. (canceled)
